(19) United States
(12) Patent Application Publication
(10) Pub. No.: US 2021/0388400 A1
(43) Pub. Date: Dec. 16, 2021

(54) METHOD OF PRODUCING TARGET SUBSTANCE FROM STARTING SUBSTANCE VIA NADH-ACCUMULATING REACTION PATHWAY

(71) Applicant: Green Earth Institute Co., Ltd., Tokyo (JP)

(72) Inventor: Toru NAKAYASHIKI, Chiba (JP)

(73) Assignee: Green Earth Institute Co., Ltd., Tokyo (JP)

(21) Appl. No.: 17/287,522

(22) PCT Filed: Oct. 24, 2018
(86) PCT No.: PCT/JP2018/039538

§ 371 Date: Apr. 22, 2021

(51) Int. Cl. C12P 7/50; C12P 7/54; C12N 9/04; C12N 9/88; C12N 9/02; C12N 9/10

(52) U.S. Cl. CPC C12P 7/50; C12P 7/54; C12N 9/0006; C12N 9/88; C12Y 203/01054; C12N 9/1029; C12Y 401/01031; C12Y 102/03003; C12N 9/0008

(57) ABSTRACT

Provided is a method of producing a target substance from a starting substance via an NADH-accumulating reaction pathway, the method comprising: incubating bacteria under an aerobic condition; and subsequently incubating the bacteria under an anaerobic condition in the presence of the starting substance and nitrate ion to produce the target substance.

### TECHNICAL FIELD

The present invention relates to a method for producing a target substance from a starting substance via an NADH-accumulating reaction pathway.

### BACKGROUND ART

A fermentation technique, in which proliferation culture of bacteria is carried out under aerobic conditions and production of substances is carried out thereafter under anaerobic conditions, is currently performed mainly using bacteria of the genus Corynebacterium (Inui M, Murakami S, Okino S, Kawaguchi H, Vertes A. A., Yukawa H, J Mol Microbiol Biotechnol. 2004; 7(4): 182-196). Such a fermentation technique has also been performed using Escherichia coli (Vemuri G. N., Eiteman M. A., Altman E., Appl Environ Microbiol. 2002 April; 68(4): 1715-1727). The above-mentioned fermentation technique is excellent from the viewpoint that bacterial cells can be prepared in a high concentration since bacteria proliferate under aerobic conditions, and from the viewpoint that production of substances can be efficiently carried out since bacterial proliferation substantially does not occur under anaerobic conditions.

### SUMMARY OF INVENTION

An objective of the present invention is to provide a method for efficiently producing a target substance from a starting substance via an NADH-accumulating reaction pathway using bacteria.

According to one aspect, the following method is provided.

A method for producing a target substance from a starting substance via an NADH-accumulating reaction pathway, the method including:

incubating bacteria under aerobic conditions; and

subsequently incubating the bacteria under anaerobic conditions in the presence of the starting substance and a nitrate ion to produce the target substance.

According to the present invention, a method for efficiently producing a target substance from a starting substance via an NADH-accumulating reaction pathway using bacteria is provided.

### BRIEF DESCRIPTION OF DRAWINGS

FIG. 1 is a schematic diagram showing a process in which coryneform bacteria produces pyruvic acid or a compound biosynthesized through pyruvic acid serving as a metabolic intermediate.

FIG. 2 is a schematic diagram showing a process in which Escherichia coli produces pyruvic acid or a compound biosynthesized through pyruvic acid serving as a metabolic intermediate.

### DESCRIPTION OF EMBODIMENTS

Hereinafter, the present invention will be described in detail, but the following description is intended to explain the present invention in detail and is not intended to limit the present invention.

### 1. Method for Producing Target Substance

Regarding the reason why efficiency of producing pyruvic acid is hindered when producing pyruvic acid from glucose under anaerobic conditions using a glycolytic system of bacteria, the inventors of the present invention have speculated as follows. In production of pyruvic acid from glucose via a glycolytic reaction pathway, 2 mol of NADH is produced from 1 mol of glucose, but since there is no reaction that consumes NADH, NADH accumulates in bacterial cells. However, in production of substances under anaerobic conditions, an amount of dissolved oxygen in a culture solution is small, and therefore NADH produced by bacteria cannot be oxidized by oxygen. NADH accumulating in bacterial cells inhibits the activity of an enzyme (GAPDH) that produces NADH, and hinders smooth progress of a glycolytic reaction. As a result, efficiency of producing pyruvic acid is hindered.

Based on the above speculation, the inventors of the present have found that efficiency of producing of pyruvic acid can be improved when a reaction of consuming NADH under nitrate respiration by adding nitrate salts is performed together with production of pyruvic acid.

In addition, regarding a compound biosynthesized through pyruvic acid serving as a metabolic intermediate (hereinafter, also referred to as pyruvic acid derivatives), the inventors of the present invention have confirmed that, in a case where NADH accumulates in bacterial cells in a reaction pathway through which pyruvic acid derivatives are produced from glucose, NADH is consumed under nitrate respiration by adding nitrate salts, and thereby efficiency of producing pyruvic acid derivatives can be improved.

The above-mentioned effect of improving efficiency of producing pyruvic acid or pyruvic acid derivatives is based on a case in which NADH is consumed under nitrate respiration by adding nitrate salts, and thereby the accumulation of NADH in bacterial cells is removed. In this manner, all activities of the enzyme inhibited by the accumulation of NADH can be restored. Accordingly, the present invention can be generalized not only to the production of pyruvic acid or pyruvic acid derivatives, but also to “any cases of producing a target substance from a starting substance via an NADH-accumulating reaction pathway.”

That is, according to the generalized invention, the following method is provided.

A method for producing a target substance from a starting substance via an NADH-accumulating reaction pathway, the method including:

incubating bacteria under aerobic conditions; and

subsequently incubating the bacteria under anaerobic conditions in the presence of the starting substance and a nitrate ion to produce the target substance.

According to this method, when the bacteria are incubated under anaerobic conditions in the presence of the starting substance and a nitrate ion, the bacteria substantially do not proliferate. Therefore, the starting substance can be efficiently used for production of substances, and NADH accumulating during the process of the production of substances can be consumed by an oxidation reaction in conjugation with a reduction reaction of the nitrate ion. Thereby, the accumulation of NADH in bacterial cells can be removed. Accordingly, the target substance can be efficiently produced.

When bacteria reduce nitrate ions, nitrite ions are produced. Nitrite ions are known to be cytotoxic. Accordingly, this method may further include reacting a nitrite ion, which is generated by reduction of the nitrate ion by the bacteria, with hydrogen peroxide such that the nitrite ion is oxidized to a nitrate ion.

Specifically, the “NADH-accumulating reaction pathway” is the following reaction pathway:

a reaction pathway which includes a reaction involving NADH production (that is, a reduction reaction of NAD to NADH) but does not include a reaction involving NADH consumption (that is, an oxidation reaction of NADH to NAD⁺); or

a reaction pathway which includes a reaction involving NADH production and a reaction involving NADH consumption, and in which a total number of NADH molecules produced in the reaction involving NADH production is greater than a total number of NADH molecules consumed in the reactions involving NADH consumption. The “total number of NADH molecules” can be rephrased as a “total number of moles of NADH molecules.”

As the former reaction pathway, a reaction pathway for producing pyruvic acid from glucose is exemplified. The reaction pathway for producing pyruvic acid from glucose includes the reaction involving NADH production, but does not include the reaction involving NADH consumption, and when one molecule of glucose is used, two molecules of NADH are produced. As the latter reaction pathway, a reaction pathway for producing acetaldehyde from glucose via acetyl-CoA is exemplified. The reaction pathway for producing acetaldehyde from glucose via acetyl-CoA includes the reaction involving NADH production and the reaction involving NADH consumption, and when one molecule of glucose is used, four molecules of NADH are produced, and two molecules of NADH are consumed.

A combination of a starting substance and a target substance is not particularly limited as long as a reaction pathway from the starting substance to the target substance is the “NADH-accumulating reaction pathway.” Examples of combinations of a starting substance and a target substance include a case in which a starting substance is glucose and a target substance is pyruvic acid, a case in which a starting substance is glucose and a target substance is acetic acid, a case in which a starting substance is glucose and a target substance is oxaloacetic acid, a case in which a starting substance is ethanol and a target substance is acetaldehyde, a case in which a starting substance is lactic acid and a target substance is pyruvic acid, a case in which a starting substance is xylose and a target substance is serine, a case in which a starting substance is succinic acid and a target substance is malic acid, and the like.

This method utilizes the known fermentation technique in which proliferation culture of bacteria is carried out under aerobic conditions, and production of substances is carried out thereafter under anaerobic conditions. Accordingly, this method can be carried out using the known fermentation technique except that the production of substances under anaerobic conditions is carried out in the presence of nitrate ions. When using the known fermentation technique, it is possible to appropriately set the type of bacteria to be used, conditions for proliferation culture, conditions for culture in production of substances, and the like, depending on the type of desired target substance to be produced. When conducting this method, descriptions in the section of “2. Method for producing pyruvic acid or pyruvic acid derivatives” to be described later can also be referred to as necessary.

In this method, the “nitrate ion” can be added into a culture solution in the form of a nitrate salt. Nitrate salts can be ionized in a culture solution, and thereby generate nitrate ions. As nitrate salts, it is possible to use, for example, ammonium nitrate, sodium nitrate, potassium nitrate, or a combination thereof.

A concentration of nitrate salts in a culture solution is preferably within a range of 10 to 500 mM, is more preferably within a range of 100 to 500 mM, and is even more preferably within a range of 200 to 300 mM, at the time when the nitrate salts are added. Furthermore, additional nitrate salts may be added according to a decrease in nitrate ions accompanying production of a target substance.

In this method, the bacteria are preferably bacteria that have been genetically engineered to not produce a fermentation substance (such as lactic acid, acetic acid, or formic acid) which is produced by the bacteria in large quantities from glucose as a raw material under anaerobic conditions, as described in the section of “2. Method for producing pyruvic acid or pyruvic acid derivatives” to be described later. In addition, the bacteria are preferably bacteria that have been genetically engineered such that a metabolic intermediate of an in vivo reaction pathway (that is, a glycolytic system) through which pyruvic acid is generated from glucose is not metabolized by reactions other than the glycolytic reaction, as described in the section of “2. Method for producing pyruvic acid or pyruvic acid derivatives” to be described later.

Accordingly, the bacteria are preferably deficient in a function of one or more genes selected from the group consisting of a gene (ldh) encoding lactate dehydrogenase, a gene (ppc) encoding phosphoenolpyruvate carboxylase, a gene (pox) encoding pyruvate oxidase, and a gene (pfl) encoding pyruvate formate lyase.

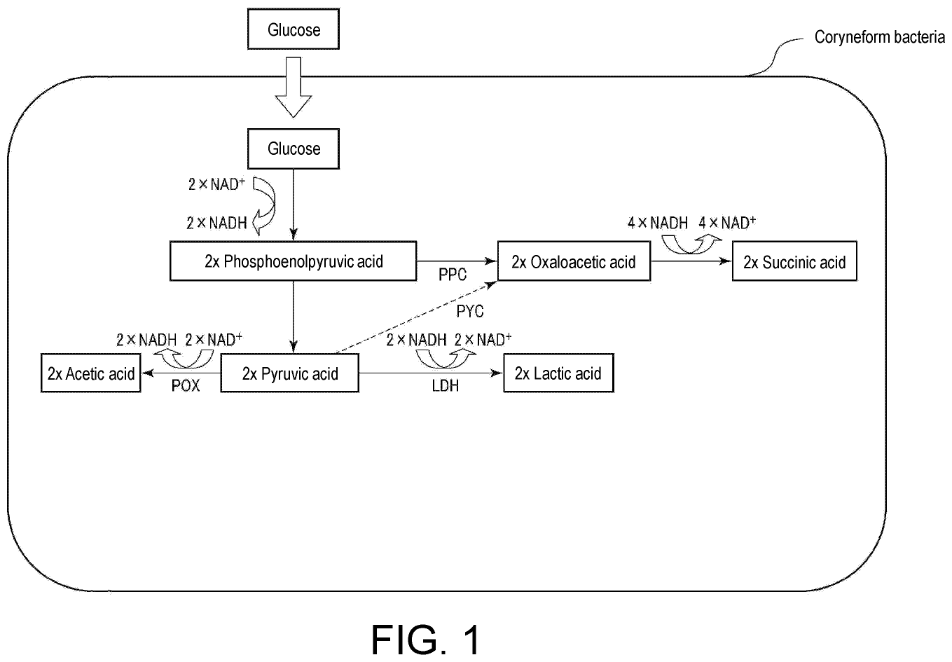

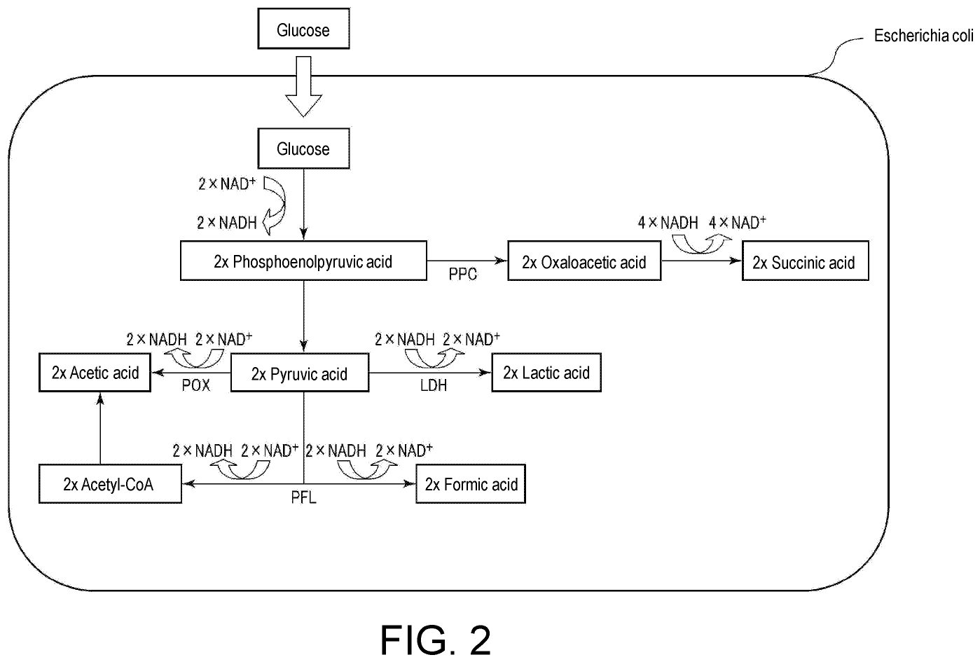

When the bacteria are deficient in a function of any gene among the above-mentioned genes, then each reaction shown in FIGS. 1 and 2 cannot proceed, and this makes it possible to efficiently direct a saccharide added as a starting substance to production of a target substance, as described in the section of “2. Method for producing pyruvic acid or pyruvic acid derivatives” to be described later. As a result, such genetically engineered bacteria can improve efficiency of producing a target substance as compared with bacteria that are not deficient in a function of the gene.

When the bacteria are deficient in a function of any gene among the above-mentioned genes, a starting substance is preferably a “saccharide,” and a target substance is preferable “pyruvic acid” or a “compound biosynthesized through a glycolytic metabolite serving as a metabolic intermediate.”

As the “saccharide,” it is possible to use a saccharide described in the section of “2. Method for producing pyruvic acid or pyruvic acid derivatives” to be described later. Examples of saccharides include monosaccharides such as fructose or glucose, disaccharides such as sucrose, polysaccharides such as a polysaccharide containing glucose as constituent monosaccharides, or a saccharified solution obtained by treating a plant (for example, non-edible agricultural waste or energy crops) with a saccharification enzyme.

The “compound biosynthesized through a glycolytic metabolite serving as a metabolic intermediate” is a compound biosynthesized through a “glycolytic metabolite” which serves as a metabolic intermediate and is selected from the group consisting of glucose-6-phosphate, fructose-6-phosphate, fructose-1,6-bisphosphate, dihydroxyacetone phosphate, glyceraldehyde-3-phosphate, 1,3-bisphosphoglyceric acid, 3-phosphoglyceric acid, phosphoenolpyruvic acid, and pyruvic acid. Examples of the “compound biosynthesized through a glycolytic metabolite serving as a metabolic intermediate” include serine biosynthesized through 3-phosphoglyceric acid serving as a metabolic intermediate, oxaloacetic acid biosynthesized through phosphoenolpyruvic acid serving as a metabolic intermediate, acetic acid biosynthesized through pyruvic acid serving as a metabolic intermediate, and acetaldehyde biosynthesized through pyruvic acid serving as a metabolic intermediate.

In a specific embodiment, a starting substance can be a “saccharide,” and a target substance can be “pyruvic acid” or a “compound biosynthesized through a glycolytic metabolite serving as a metabolic intermediate.” That is, according to this embodiment, the following method is provided.

A method for producing, from a saccharide via an NADH-accumulating reaction pathway, “pyruvic acid” or a “compound biosynthesized through a glycolytic metabolite serving as a metabolic intermediate,” the method including:

incubating bacteria under aerobic conditions; and

subsequently incubating the bacteria under anaerobic conditions in the presence of a saccharide and a nitrate ion to produce “pyruvic acid” or a “compound biosynthesized through a glycolytic metabolite serving as a metabolic intermediate.”

### 2. Method for Producing Pyruvic Acid or Pyruvic Acid Derivatives

Hereinafter, the present invention will be described with a case in which a starting substance is a “saccharide” and a target substance is “pyruvic acid” or a “compound biosynthesized through pyruvic acid serving as a metabolic intermediate (hereinafter, also referred to as pyruvic acid derivatives)” as an example. As described above, the concept of the present invention is to consume NADH by a reduction reaction of nitrate ions to remove the accumulation of NADH in bacterial cells, and thereby efficiently produce a “substance produced via an NADH-accumulating reaction pathway.” Accordingly, the present invention is not limited to the production of pyruvic acid or pyruvic acid derivatives. The following description is provided for the purpose of assisting the understanding of the present invention.

### 2-1. First Embodiment

According to a first embodiment, a method for producing, from a saccharide via an NADH-accumulating reaction pathway, pyruvic acid or a compound biosynthesized through pyruvic acid serving as a metabolic intermediate, includes:

incubating bacteria under aerobic conditions; and

subsequently incubating the bacteria under anaerobic conditions in the presence of a saccharide and a nitrate ion to produce pyruvic acid or a compound biosynthesized through pyruvic acid serving as a metabolic intermediate.

In this method, as literally defined, the “pyruvic acid” is produced from a saccharide via an NADH-accumulating reaction pathway, and the “compound biosynthesized through pyruvic acid serving as a metabolic intermediate” is produced from a saccharide through pyruvic acid via an NADH-accumulating reaction pathway. Accordingly, in the present specification, the term “compound biosynthesized through pyruvic acid serving as a metabolic intermediate” does not refer to “any compound produced from a saccharide through pyruvic acid,” but refers to a “compound produced from a saccharide through pyruvic acid via an NADH-accumulating reaction pathway.” The “compound biosynthesized through pyruvic acid serving as a metabolic intermediate” is, for example, acetic acid and acetaldehyde.

This method utilizes the known fermentation technique in which proliferation culture of bacteria is carried out under aerobic conditions, and production of substances is carried out thereafter under anaerobic conditions.

<Bacteria>

As the bacteria, it is possible to use any bacteria having an in vivo reaction pathway (that is, a glycolytic system) in which glucose is degraded to pyruvic acid. Bacteria are generally aerobic bacteria, such as coryneform bacteria or Escherichia coli.

Coryneform bacteria are a group of microorganisms defined in Bargeys Manual of Determinative Bacteriology, Vol. 8, 599 (1974), and they are not particularly limited as long as they proliferate under normal aerobic conditions. Specific examples thereof include bacteria of the genus Corynebacterium, bacteria of the genus Brevibacterium, bacteria of the genus Arthrobacter, bacteria of the genus Mycobacterium, bacteria of the genus Micrococcus, and the like. Among coryneform bacteria, bacteria of the genus Corynebacterium are preferable.

Examples of bacteria of the genus Corynebacterium include Corynebacterium glutamicum, Corynebacterium efficiens, Corynebacterium ammoniagenes, Corynebacterium halotolerance, Corynebacterium alkanolyticum, and the like. Among them, Corynebacterium glutamicum is preferable. Examples of suitable strains include Corynebacterium glutamicum strain R (FERM P-18976), strain ATCC 13032, strain ATCC 13869, strain ATCC 13058, strain ATCC 13059, strain ATCC 13060, strain ATCC 13232, strain ATCC 13286, strain ATCC 13287, strain ATCC 13655, strain ATCC 13745, strain ATCC 13746, strain ATCC 13761, strain ATCC 14020, strain ATCC 31831, MJ-233 (FERM BP-1497), MJ-233AB-41 (FERM BP-1498), and the like. Among them, strain R (FERM P-18976), strain ATCC 13032, and strain ATCC 13869 are preferable.

According to the molecular biological classification, coryneform bacteria such as Brevibacterium flavum, Brevibacterium lactofermentum, Brevibacterium divaricatum, and Corynebacterium lilium are unified under the bacterial name, Corynebacterium glutamicum [Liebl, W. et al., Transfer of Brevibacterium divaricatum DSM 202971, “Brevibacterium flavum” DSM 20411, “Brevibacterium lactofermentum” DSM 20412 and DSM 1412, and Corynebacterium glutamicum and their distinction by rRNA gene restriction patterns. Int J Syst Bacteriol. 41:255-260. (1991); Kazuo Komagata et al., Classification of Coryneform Bacteria, Fermentation and Industry, 45:944-963 (1987)].

Brevibacterium lactofermentum strain ATCC 13869, Brevibacterium flavum strains MJ-233 (FERM BP-1497) and MJ-233AB-41 (FERM BP-1498), and the like in the old classification are also suitable Corynebacterium glutamicum.

Examples of bacteria of the genus Brevibacterium include Brevibacterium ammoniagenes (for example, strain ATCC 6872), and the like.

Examples of bacteria of the genus Arthrobacter include Arthrobacter globiformis (for example, strains ATCC 8010, ATCC 4336, ATCC 21056, ATCC 31250, ATCC 31738, and ATCC 35698), and the like.

Examples of bacteria of the genus Mycobacterium include Mycobacterium bovis (for example, strains ATCC 19210 and ATCC 27289), and the like.

Examples of bacteria of the genus Micrococcus include Micrococcus freudenreichii (for example, strain No. 239 (FERM P-13221)), Micrococcus lueteus (for example, strain No. 240 (FERM P-13222)), Micrococcus ureae (for example, strain IAM 1010), Micrococcus roseus (for example, strain IFO3764), and the like.

The bacteria are preferably bacteria that have been genetically engineered to not produce a fermentation substance (such as lactic acid, acetic acid, or formic acid) which is produced by the bacteria in large quantities from glucose as a raw material under anaerobic conditions. Examples of such genetically engineered bacteria include the following bacteria:

bacteria that have been genetically engineered to be deficient in a function of a gene (ldh) encoding lactate dehydrogenase involved in lactic acid fermentation in a case where bacteria (before being genetically engineered) are bacteria (such as coryneform bacteria and Escherichia coli) which produce lactic acid as a fermentation substance under anaerobic conditions;

bacteria that have been genetically engineered to be deficient in a function of a gene (pox) encoding pyruvate oxidase involved in acetic acid fermentation in a case where bacteria (before being genetically engineered) are bacteria (such as coryneform bacteria) which produce acetic acid as a fermentation substance under anaerobic conditions; and

bacteria that have been genetically engineered to be deficient in a function of a gene (pfl) encoding pyruvate formate lyase involved in formic acid fermentation in a case where bacteria (before being genetically engineered) are bacteria (such as Escherichia coli) which produce formic acid as a fermentation substance under anaerobic conditions.

When the bacteria have isozymes for the above-mentioned enzymes, it is sufficient for the bacteria to be genetically engineered such that they are deficient in a function of the gene encoding one of the enzymes.

In addition, the bacteria are preferably bacteria that have been genetically engineered such that a metabolic intermediate of an in vivo reaction pathway (that is, a glycolytic system) through which pyruvic acid is generated from glucose is not metabolized by reactions other than the glycolytic reaction. Examples of such genetically engineered bacteria include bacteria that have been genetically engineered to be deficient in a function of a gene (ppc) encoding phosphoenolpyruvate carboxylase so that phosphoenolpyruvic acid (a precursor of pyruvic acid in the glycolytic system) is not metabolized to oxaloacetic acid.

Accordingly, the bacteria are preferably deficient in a function of one or more genes selected from the group consisting of a gene (ldh) encoding lactate dehydrogenase, a gene (ppc) encoding phosphoenolpyruvate carboxylase, a gene (pox) encoding pyruvate oxidase, and a gene (pfl) encoding pyruvate formate lyase.

Bacteria deficient in a function of the gene (ldh) encoding lactate dehydrogenase (LDH) cannot convert pyruvic acid to lactic acid (refer to “LDH” shown in FIGS. 1 and 2). Bacteria deficient in a function of the gene (ppc) encoding phosphoenolpyruvate carboxylase (PPC) cannot convert phosphoenolpyruvic acid to oxaloacetic acid (refer to “PPC” shown in FIGS. 1 and 2). Bacteria deficient in a function of the gene (pox) encoding pyruvate oxidase (POX) cannot convert pyruvic acid to acetic acid (refer to “POX” shown in FIGS. 1 and 2). Bacteria deficient in a function of the gene (pfl) encoding pyruvate formate lyase (PFL) cannot convert pyruvic acid to formic acid (refer to “PFL” shown in FIG. 2).

Accordingly, when the bacteria are deficient in a function of any gene among the above-mentioned genes, then each reaction shown in FIGS. 1 and 2 cannot proceed, and this makes it possible to efficiently direct a saccharide added as a starting substance to production of pyruvic acid or pyruvic acid derivatives. As a result, such genetically engineered bacteria can improve efficiency of producing pyruvic acid or pyruvic acid derivatives as compared with bacteria that are not deficient in a function of the gene.

In the present specification, the phrase, “deficient in a function of a gene” means that a function of this gene is deficient by genetic engineering, and also means that a function of this gene is deficient because bacteria naturally do not contain the gene.

In addition, in the present specification, the sentence, “a function of this gene is deficient by genetic engineering” means as follows:

an enzyme protein is not expressed or a gene expression product does not function as an enzyme by modification (for example, deletion, substitution, and/or insertion) of a part or all of the gene; and

an enzyme protein is not expressed by modification (for example, deletion, substitution, and/or insertion) of a part or all of a promoter of the gene.

In the present specification, bacteria deficient in a function of a gene by deleting a part or all of the gene by genetic engineering are referred to as a “deletion strain.” The deletion strain can be produced according to a known method. For example, the deletion strain can be produced as follows: a deletion type gene modified so as not to produce an enzyme protein encoded by a target gene is produced, a wild-type strain is transformed with DNA containing the deletion type gene, and homologous recombination is caused between the deletion type gene and a gene on the chromosome of the wild-type strain.

The bacteria may be a deletion strain deficient in a gene (ldh) encoding lactate dehydrogenase, may be a deletion strain deficient in a gene (ldh) encoding lactate dehydrogenase and a gene (ppc) encoding phosphoenolpyruvate carboxylase, or may be a deletion strain deficient in a gene (ldh) encoding lactate dehydrogenase, a gene (ppc) encoding phosphoenolpyruvate carboxylase, and a gene (pox) encoding pyruvate oxidase. These deletion strains are preferably deletion strains of coryneform bacteria.

Alternatively, the bacteria may be a deletion strain deficient in a gene (pfl) encoding pyruvate formate lyase, or may be a deletion strain deficient in a gene (pfl) encoding pyruvate formate lyase and a gene (ldh) encoding lactate dehydrogenase. These deletion strains are preferably deletion strains of Escherichia coli.

<Incubation Under Aerobic Conditions>

The bacteria are first incubated under aerobic conditions. Accordingly, the bacteria can proliferate, and bacterial cells can be prepared at a high concentration. Therefore, in the following description, incubation under aerobic conditions is also referred to as “proliferation culture.”

The term “aerobic conditions” specifically refers to a condition in which oxygen is supplied to a culture solution in an amount sufficient for bacterial proliferation over the incubation period. The aerobic conditions can be realized by, for example, shaking a container accommodating a bacteria-containing culture solution in an air atmosphere. Alternatively, the aerobic conditions can be realized by, for example, agitating a bacteria-containing culture solution while aerating the bacteria-containing culture solution with air.

As a culture solution used in the proliferation culture, it is possible to use a culture solution suitable for bacterial proliferation. Specifically, it is possible to use a natural medium or synthetic medium containing a carbon source, a nitrogen source, inorganic salts, and the like. In the present specification, a concentration of each of components in the culture solution refers to a final concentration thereof in the culture solution at a time point when each of the components is added.

As the “carbon source,” it is possible to use any substance as long as it is a substance that can be used as a nutrient source by bacteria. Examples of carbon sources include saccharides (for example, monosaccharides such as glucose, fructose, mannose, xylose, arabinose, and galactose; disaccharides such as sucrose, maltose, lactose, cellobiose, xylobiose, and trehalose; polysaccharides such as cellulose and starch; and molasses); sugar alcohols such as mannitol, sorbitol, xylitol, and glycerin; organic acids such as acetic acid, citric acid, lactic acid, fumaric acid, maleic acid, and gluconic acid; alcohols such as ethanol, propanol, and ribitol; and hydrocarbons such as normal paraffin. For the carbon source, one kind may be used, or two or more kinds may be mixed and used. A concentration of the carbon source in the culture solution can be generally set to about 0.1 to about 30 (w/v) %, and preferably about 1 to about 10 (w/v) %. In addition, additional a carbon source may be added according to a decrease in carbon source accompanying culture.

As the “nitrogen source,” it is possible to use, for example, inorganic or organic ammonium salts such as ammonium chloride, ammonium sulfate, ammonium nitrate, sodium nitrate, potassium nitrate, and ammonium acetate; urea; aqueous ammonia, and the like. In addition, it is also possible to use protein hydrolysates such as corn steep liquor, meat extract, yeast extract, peptone, NZ-amine, soybean hydrolysate, and casein hydrolysate, and nitrogen-containing organic compounds such as amino acids. For the nitrogen source, one kind may be used, or two or more kinds may be mixed and used. A concentration of the nitrogen source in the culture solution varies depending on a nitrogen compound used, but it can be generally set to about 0.1 to about 10 (w/v) %.

As the “inorganic salts,” it is possible to use, for example, phosphate salts, sulfate salts, and salts of metal such as magnesium, potassium, manganese, iron, and zinc. Specifically, it is possible to use potassium dihydrogen phosphate, potassium monohydrogen phosphate, magnesium sulfate, sodium chloride, ferrous nitrate, iron(II) sulfate, manganese sulfate, zinc sulfate, cobalt sulfate, calcium carbonate, and the like. For the inorganic salts, one kind may be used, or two or more kinds may be mixed and used. A concentration of the inorganic salts in the culture solution varies depending on inorganic salts used, but it can be generally set to about 0.01 to about 1 (w/v) %.

In addition, nutritional substances can be added as necessary. Examples of nutritional substances include meat extract, peptone, polypeptone, yeast extract, dried yeast, corn steep liquor, skim milk powder, defatted soybean hydrochloric acid hydrolysate, extracts of animal and plant or microbial cells and their decomposed products, and the like. A concentration of the nutritional substance in the culture solution varies depending on a nutritional substance used, but it can be generally set to about 0.1 to about 10 (w/v) %.

In addition, as necessary, it is possible to add factors that promote bacterial proliferation, specifically vitamins, nucleotides, or amino acids. Examples of vitamins include biotin, thiamine (vitamin B1), pyridoxine (vitamin B6), pantothenic acid, inositol, and nicotinic acid. These vitamins can be substituted by meat extract, yeast extract, corn steep liquor, casamino acid, and the like. Furthermore, it is possible to add a commercially available anti-foaming agent to the culture solution in an appropriate amount in order to suppress foaming during a culture period.

In addition, appropriate antibiotics can be added as necessary. Examples of antibiotics include kanamycin and spectinomycin.

A pH of the culture solution may be any pH as long as it is suitable for bacterial proliferation. A pH of the culture solution can be set, for example, within a range of 5 to 9, preferably within a range of 6 to 8. A pH of the culture solution can be adjusted using a pH buffer solution containing an inorganic or organic acid, an alkaline solution such as an aqueous solution of potassium hydroxide, urea, calcium carbonate, ammonia, 4-morpholinopropanesulfonic acid, or the like. A pH of the culture solution may be adjusted during proliferation culture as necessary.

The proliferation culture can be carried out at a temperature suitable for bacterial proliferation and for a period required for bacterial proliferation. As a culture temperature in the proliferation culture, 20° C. to 42° C. is suitable in general, and it is preferably 30° C. to 39° C. A culture time in the proliferation culture is generally 1 to 6 days.

<Incubation Under Anaerobic Conditions>

After the proliferation culture, the bacteria are incubated under anaerobic conditions in the presence of a saccharides and a nitrate ion. Accordingly, the bacteria can produce pyruvic acid or pyruvic acid derivatives from the saccharide. Therefore, in the following description, incubation under anaerobic conditions is also referred to as “production culture.”

The bacteria substantially do not proliferate when they are put under anaerobic conditions, and therefore, a saccharide can be efficiently used as a raw material for production of substances, instead of being used as an energy source for the proliferation. In addition, when the bacteria are put in the presence of a nitrate ion, the nitrate ion is reduced to a nitrite ion by Reaction Formula (1), and NADH can be consumed during this reaction.

NO₃⁻+NADH+H⁺→NO₂⁻+NAD⁺H₂O (1)

Through this reaction, in the bacteria, NADH generated in the process of producing pyruvic acid from glucose is consumed, and the accumulation of NADH in bacterial cells is removed. Accordingly, it is possible to promote incorporation of a saccharide into bacterial cells and to promote the reaction of producing pyruvic acid from the saccharide.

Accordingly, by performing the production culture in the presence of a saccharide and a nitrate ion under anaerobic conditions, the bacteria can efficiently produce pyruvic acid or pyruvic acid derivatives from the saccharide.

Furthermore, a nitrite ion (NO₂⁻) is known to exhibit cell cytotoxicity, and by adding a hydrogen peroxide solution, nitrite ions accumulated by the reaction of Reaction Formula (1) can be returned to nitrate ions (NO₃⁻) again by Reaction Formula (2).

NO₂⁻+H₂O₂→NO₃+H₂O (2)

That is, by using hydrogen peroxide, the cycle consisting of Reaction Formula (1) and Reaction Formula (2) can be permanently repeated. In consideration of an influence of hydrogen peroxide on cells, the reaction of Reaction Formula (2) is preferably carried out in a state where bacteria are separated from the culture solution in the production culture. Specifically, at the timing when a concentration of nitrite ions in a culture solution increases during the production culture, bacteria can be separated and removed from the culture solution, a hydrogen peroxide solution can be added into the culture solution not containing the bacteria and reacted for a predetermined time, and thereafter, the separated and removed bacteria can be returned to the culture solution to continue the production culture.

Accordingly, the method for producing pyruvic acid or pyruvic acid derivatives may further include reacting a nitrite ion, which is generated by reduction of the nitrate ion by the bacteria, with hydrogen peroxide such that the nitrite ion is oxidized to a nitrate ion.

Hereinafter, culture conditions for the production culture will be described in detail.

The production culture is carried out under anaerobic conditions in the presence of a saccharide and a nitrate ion.

“Incubating bacteria in the presence of a saccharide and a nitrate ion” can be performed by incubating the bacteria in a culture solution containing the saccharide and the nitrate ion. More specifically, “incubating bacteria in the presence of a saccharide and a nitrate ion” can be performed by incubating the bacteria in a culture solution into which the saccharide and a nitrate salt are added.

The term “anaerobic conditions” specifically refers to a condition in which oxygen is not actively supplied to a bacteria-containing culture solution over the incubation period. More specifically, the term anaerobic conditions refers to a condition in which a concentration of dissolved oxygen in the culture solution is within a range of 0 to 2 ppm, preferably refers to a condition in which a concentration of dissolved oxygen in the culture solution is within a range of 0 to 1 ppm, and more preferably refers to a condition in which a concentration of dissolved oxygen in the culture solution is within a range of 0 to 0.5 ppm. A concentration of dissolved oxygen in the culture solution can be measured using, for example, a dissolved oxygen meter.

Strictly speaking, the term “anaerobic conditions” is a technical term that refers to a condition in which dissolved oxygen is not present in the culture solution. On the other hand, the “anaerobic conditions” adopted in the production culture may be a condition in which presence of a small amount of dissolved oxygen in the culture solution is allowed as long as bacterial proliferation can be inhibited, and efficiency of producing pyruvic acid or pyruvic acid derivatives can be increased. Accordingly, the term “anaerobic conditions” in the present specification includes not only “a condition in which dissolved oxygen is not present in the culture solution” but also “a condition in which a small amount of dissolved oxygen is present in the culture solution.”

The anaerobic conditions can be realized by, for example, allowing a bacteria-containing culture solution to stand in an air atmosphere without shaking it, and thereby reducing an amount of dissolved oxygen in the culture solution after a lapse of a predetermined time. Alternatively, the anaerobic conditions can be realized by, for example, aerating a bacteria-containing culture solution with an inert gas, specifically a nitrogen gas. Alternatively, the anaerobic conditions can be realized by, for example, sealing a container accommodating a bacteria-containing culture solution.

The production culture is preferably carried out in a state in which the bacteria are suspended in the culture solution at a high density. When the bacteria are cultured in such a state, it is possible to more efficiently produce pyruvic acid or pyruvic acid derivatives.

The “state in which the bacteria are suspended in the culture solution at a high density” refers to, for example, a state in which the bacteria are suspended in the culture solution such that a weight % of wet bacterial cells of the bacteria is within a range of 1 to 50 (w/v) %. The “state in which the bacteria are suspended in the culture solution at a high density” preferably refers to a state in which the bacteria are suspended in the culture solution such that a weight % of wet bacterial cells of the bacteria is within a range of 3 to 30 (w/v) %.

The production culture is preferably carried out in the state in which the bacteria are suspended in the culture solution at a high density, but because it is preferable to carry out the proliferation culture in an environment in which the bacteria can easily proliferate, the proliferation culture is preferably carried out in a state in which the bacteria are suspended in the culture solution at a density lower than the above-mentioned density adopted for the production culture.

The production culture can be carried out after the culture solution is exchanged after the proliferation culture. Specifically, the production culture can be carried out as follows: after the proliferation culture, a container containing the culture solution and the bacteria suspended in the culture solution is subjected to a centrifuge machine to precipitate the bacteria; thereafter, the culture solution for proliferation culture is removed from the container; and the separated bacteria are suspended in a culture solution for production culture.

Alternatively, the production culture can be continuously carried out without exchanging the culture solution by adding additional components (that is, a saccharide and nitrate salts) required for the production culture in the bacteria-containing culture solution after the proliferation culture.

The production culture can be carried out in a culture solution containing a saccharide and a nitrate ion. In the present specification, a concentration of each of components in the culture solution refers to a final concentration thereof in the culture solution at a time point when each of the components is added.

As the “saccharide,” any saccharide can be used as long as it is a saccharide that can be incorporated into a living body by bacteria and can be converted to pyruvic acid. Specific examples of saccharides include monosaccharides such as glucose, fructose, mannose, xylose, arabinose, and galactose; disaccharides such as sucrose, maltose, lactose, cellobiose, xylobiose, and trehalose; polysaccharides such as starch; molasses; and the like. Among them, monosaccharides are preferable, fructose and glucose are more preferable, and glucose is even more preferable. Furthermore, a saccharide containing glucose as the constituent monosaccharide is also preferable. Specifically, a disaccharide such as sucrose, an oligosaccharide containing glucose as the constituent monosaccharide, and a polysaccharide containing glucose as the constituent monosaccharide are also preferable. For the saccharide, one kind may be used, or two or more kinds may be mixed and used. In addition, the saccharide can be added into the culture solution in the form of a saccharified solution (containing a plurality types of saccharides such as glucose and xylose) obtained by saccharifying, with a saccharification enzyme or the like, non-edible agricultural waste such as rice straw, bagasse, and corn stover, and energy crops such as switchgrass, napier grass, and miscanthus.

A concentration of the saccharide in the culture solution can be set as high as possible within a range not inhibiting the production of pyruvic acid or pyruvic acid derivatives. A concentration of the saccharide in the culture solution is preferably within a range of about 0.1 to about 40 (w/v) %, and is more preferably within a range of about 1 to about 20 (w/v) %. In addition, additional saccharides may be added according to a decrease in saccharide accompanying the production of pyruvic acid or pyruvic acid derivatives.

The “nitrate ion” can be added into the culture solution in the form of a nitrate salt. Nitrate salts can be ionized in the culture solution, and thereby generate nitrate ions. As nitrate salts, it is possible to use, for example, ammonium nitrate, sodium nitrate, potassium nitrate, or a combination thereof.

A concentration of nitrate salts in the culture solution is preferably within a range of 10 to 500 mM, is more preferably within a range of 100 to 500 mM, and is even more preferably within a range of 200 to 300 mM. In addition, additional nitrate salts may be added according to a decrease in nitrate ions accompanying the production of pyruvic acid or pyruvic acid derivatives.

The “culture solution containing a saccharide and a nitrate ion” may contain other required components. Examples of the other required components include a carbon source other than saccharides, a nitrogen source other than nitrate salts, inorganic salts, and the like.

Examples of the “carbon source other than saccharides” include sugar alcohols such as mannitol, sorbitol, xylitol, and glycerin; organic acids such as acetic acid, citric acid, lactic acid, fumaric acid, maleic acid, and gluconic acid; alcohols such as ethanol, propanol, and ribitol; hydrocarbons such as normal paraffin; salts containing carbon dioxide and carbonate ions; and the like. For the carbon source other than saccharides, one kind may be used, or two or more kinds may be mixed and used. A concentration of the carbon source other than saccharides in the culture solution can be generally set to about 0.1 to about 20 (w/v) %.

Examples of the “nitrogen source other than nitrate salts” include inorganic or organic ammonium compounds such as ammonium chloride, ammonium sulfate, and ammonium acetate, urea, aqueous ammonia, and the like. In addition, as the nitrogen source other than nitrate salts, it is also possible to use protein hydrolysates such as corn steep liquor, meat extract, yeast extract, peptone, NZ-amine, soybean hydrolysate, and casein hydrolysate, and nitrogen-containing organic compounds such as amino acids. For the nitrogen source other than nitrate salts, one kind may be used, or two or more kinds may be mixed and used. A concentration of the nitrogen source other than nitrate salts in the culture solution varies depending on a nitrogen compound used, but it can be generally set to about 0.1 to about 10 (w/v) %.

As the “inorganic salts,” it is possible to use, for example, phosphate salts, sulfate salts, and salts of metal such as magnesium, potassium, manganese, iron, and zinc. Specific examples thereof include potassium dihydrogen phosphate, potassium monohydrogen phosphate, magnesium sulfate, sodium chloride, iron(II) sulfate, manganese sulfate, zinc sulfate, cobalt sulfate, calcium carbonate, and the like. For the inorganic salts, one kind may be used, or two or more kinds may be mixed and used. A concentration of the inorganic salts in the culture solution varies depending on inorganic salts used, but it can be generally set to about 0.01 to about 1 (w/v) %.

Furthermore, the culture solution may contain vitamins as necessary. Examples of vitamins include biotin, thiamine (vitamin B1), pyridoxine (vitamin B6), pantothenic acid, inositol, nicotinic acid, and the like.

A pH of the culture solution may be any pH as long as it is suitable for the production of pyruvic acid or pyruvic acid derivatives. A pH of the culture solution can be preferably set within a range of 6 to 8. A pH of the culture solution can be adjusted using a pH buffer solution containing an inorganic or organic acid, an alkaline solution such as an aqueous solution of potassium hydroxide, urea, calcium carbonate, ammonia, 4-morpholinopropanesulfonic acid, or the like. A pH of the culture solution may be adjusted during the production culture as necessary.

The production culture can be carried out at a temperature suitable for the production of pyruvic acid or pyruvic acid derivatives over a period required for the production of pyruvic acid or pyruvic acid derivatives. A culture temperature in the production culture is preferably about 20° C. to about 50° C., and is more preferably about 25° C. to about 47° C. A culture time in the production culture is preferably about 3 hours to 7 days, and is more preferably about 1 to 3 days.

The production culture may be a batch type, a fed-batch type, or a continuous type. Among them, the batch type is preferable.

<Recovery of Pyruvic Acid or Pyruvic Acid Derivatives>

When the proliferation culture and the subsequent production culture are carried out according to the first embodiment described above, pyruvic acid or pyruvic acid derivatives are produced in the culture solution.

The pyruvic acid or pyruvic acid derivatives can be recovered by recovering the culture solution after the production culture, but the pyruvic acid or pyruvic acid derivatives can be further separated and purified from the culture solution by a known method. Examples of such known methods include a distillation method, a concentration method, an ion exchange resin method, an activated carbon adsorption and elution method, a solvent extraction method, a crystallization method, and the like. An appropriate separation and purification method can be selected according to the type of desired substance to be recovered. For example, pyruvic acid can be separated and purified by a solvent extraction method. For example, acetic acid can be separated and purified by a distillation method.

<Reaction Pathway of Coryneform Bacteria>

FIG. 1 shows the process in which a coryneform bacterium produces pyruvic acid or pyruvic acid derivatives. In FIG. 1, glucose is used as an example of a saccharide.

As shown in FIG. 1, glucose incorporated into the coryneform bacterium is converted to pyruvic acid through the glycolytic system. Two moles of pyruvic acid are produced from one mole of glucose, and in this production process, two moles of NADH are produced. The process in which pyruvic acid is produced from glucose does not include a reaction involving NADH consumption. Accordingly, NADH is overproduced and accumulated in the reaction pathway that produces pyruvic acid from glucose. It is thought that, when nitrate salts are added into a culture solution, NADH is consumed during the conversion of nitrate ions to nitrite ions, this removes the accumulation of NADH in bacterial cells, the reaction of producing pyruvic acid from glucose proceeds smoothly thereby, and as a result, efficiency of producing pyruvic acid is increased.

As shown in FIG. 1, the produced pyruvic acid is converted to acetic acid by pyruvate oxidase (POX). Two moles of acetic acid are produced from one mole of glucose, and in this production process, four moles of NADH are produced. The process in which acetic acid is produced from glucose does not include a reaction involving NADH consumption. Accordingly, NADH is overproduced and accumulated in the reaction pathway that produces acetic acid from glucose. It is thought that, when nitrate salts are added into a culture solution, NADH is consumed during the conversion of nitrate ions to nitrite ions, this removes the accumulation of NADH in bacterial cells, the reaction of producing acetic acid from glucose proceeds smoothly thereby, and as a result, efficiency of producing acetic acid is increased.

As shown in FIG. 1, the produced pyruvic acid is converted to lactic acid by lactate dehydrogenase (LDH). Two moles of lactic acid are produced from one mole of glucose, and in this production process, two moles of NADH are produced, and two moles of NADH are consumed. Accordingly, an amount of NADH produced and an amount of NADH consumed are the same in the reaction pathway that produces lactic acid from glucose. Regarding the production of lactic acid, since the overproduction of NADH does not occur, the effect of improving the production efficiency by adding nitrate salts cannot be expected.

As shown in FIG. 1, phosphoenolpyruvic acid, which is a precursor of pyruvic acid, is converted to oxaloacetic acid by phosphoenolpyruvate carboxylase (PPC) and thereafter converted to succinic acid. Two moles of succinic acid are produced from one mole of glucose, and in this production process, two moles of NADH are produced, and four moles of NADH are consumed. Accordingly, NADH is overconsumed in the reaction pathway that produces succinic acid from glucose. Regarding the production of succinic acid, since the overproduction of NADH does not occur, the effect of improving the production efficiency by adding nitrate salts cannot be expected. Succinic acid is produced by the above-mentioned reaction pathway that produces succinic acid, but it is also produced by a reaction pathway in which pyruvic acid is converted to oxaloacetic acid by pyruvate carboxylase (PYC), although this is a weakly active reaction pathway.

<Reaction Pathway of Escherichia coli>

FIG. 2 shows the process in which Escherichia coli produces pyruvic acid or pyruvic acid derivatives. In FIG. 2, glucose is used as an example of a saccharide.

As shown in FIG. 2, glucose incorporated into Escherichia coli is converted to pyruvic acid through the glycolytic system. Two moles of pyruvic acid are produced from one mole of glucose, and in this production process, two moles of NADH are produced. The process in which pyruvic acid is produced from glucose does not include a reaction involving NADH consumption. Accordingly, NADH is overproduced and accumulated in the reaction pathway that produces pyruvic acid from glucose. It is thought that, when nitrate salts are added into a culture solution, NADH is consumed during the conversion of nitrate ions to nitrite ions, this removes the accumulation of NADH in bacterial cells, the reaction of producing pyruvic acid from glucose proceeds smoothly thereby, and as a result, efficiency of producing pyruvic acid is increased.

As shown in FIG. 2, the produced pyruvic acid is converted to acetic acid by pyruvate oxidase (POX). Two moles of acetic acid are produced from one mole of glucose, and in this production process, four moles of NADH are produced. The process in which acetic acid is produced from glucose does not include a reaction involving NADH consumption. Accordingly, NADH is overproduced and accumulated in the reaction pathway that produces acetic acid from glucose. It is thought that, when nitrate salts are added into a culture solution, NADH is consumed during the conversion of nitrate ions to nitrite ions, this removes the accumulation of NADH in bacterial cells, the reaction of producing acetic acid from glucose proceeds smoothly thereby, and as a result, efficiency of producing acetic acid is increased.

As shown in FIG. 2, the produced pyruvic acid is converted to lactic acid by lactate dehydrogenase (LDH). Two moles of lactic acid are produced from one mole of glucose, and in this production process, two moles of NADH are produced, and two moles of NADH are consumed. Accordingly, an amount of NADH produced and an amount of NADH consumed are the same in the reaction pathway that produces lactic acid from glucose. Regarding the production of lactic acid, since the overproduction of NADH does not occur, the effect of improving the production efficiency by adding nitrate salts cannot be expected.

As shown in FIG. 2, the produced pyruvic acid is converted to formic acid by pyruvate formate lyase (PFL). Two moles of formic acid are produced from one mole of glucose, and in this production process, two moles of NADH are produced, and two moles of NADH are consumed. Accordingly, an amount of NADH produced and an amount of NADH consumed are the same in the reaction pathway that produces formic acid from glucose. Regarding the production of formic acid, since the overproduction of NADH does not occur, the effect of improving the production efficiency by adding nitrate salts cannot be expected. In addition, it is also known that formic acid is converted to carbon dioxide in the presence of nitric acid.

As shown in FIG. 2, phosphoenolpyruvic acid, which is a precursor of pyruvic acid, is converted to oxaloacetic acid by phosphoenolpyruvate carboxylase (PPC) and thereafter converted to succinic acid. Two moles of succinic acid are produced from one mole of glucose, and in this production process, two moles of NADH are produced, and four moles of NADH are consumed. Accordingly, NADH is overconsumed in the reaction pathway that produces succinic acid from glucose. Regarding the production of succinic acid, since the overproduction of NADH does not occur, the effect of improving the production efficiency by adding nitrate salts cannot be expected.

### 2-2. Second Embodiment

According to a second embodiment, a method for producing, from a saccharide via an NADH-accumulating reaction pathway, pyruvic acid or a compound (hereinafter, also referred to as pyruvic acid derivatives) biosynthesized through pyruvic acid serving as a metabolic intermediate, includes:

incubating bacteria under anaerobic conditions in the presence of a saccharide and a nitrate ion to produce pyruvic acid or a compound biosynthesized through pyruvic acid serving as a metabolic intermediate, where the bacteria are deficient in a function of one or more genes selected from the group consisting of a gene (ldh) encoding lactate dehydrogenase, a gene (ppc) encoding phosphoenolpyruvate carboxylase, a gene (pox) encoding pyruvate oxidase, and a gene (pfl) encoding pyruvate formate lyase.

The method according to the second embodiment includes production culture under anaerobic conditions, and may or may not include proliferation culture under aerobic conditions. The method according to the second embodiment can be carried out in the same procedure as the method according to the first embodiment, except that the above-mentioned “bacteria which are deficient in a function of one or more genes selected from the group consisting of ldh, ppc, pox, and pfl” is used.

For the “bacteria which are deficient in a function of one or more genes selected from the group consisting of ldh, ppc, pox, and pfl,” the description in the section of “2-1. First embodiment” can be referred to.

The “bacteria which are deficient in a function of one or more genes selected from the group consisting of ldh, ppc, pox, and pfl” may be a deletion strain deficient in a gene (ldh) encoding lactate dehydrogenase, may be a deletion strain deficient in a gene (ldh) encoding lactate dehydrogenase and a gene (ppc) encoding phosphoenolpyruvate carboxylase, or may be a deletion strain deficient in a gene (ldh) encoding lactate dehydrogenase, a gene (ppc) encoding phosphoenolpyruvate carboxylase, and a gene (pox) encoding pyruvate oxidase. These deletion strains are preferably deletion strains of coryneform bacteria.

Alternatively, the “bacteria which are deficient in a function of one or more genes selected from the group consisting of ldh, ppc, pox, and pfl” may be a deletion strain deficient in a gene (pfl) encoding pyruvate formate lyase, or may be a deletion strain deficient in a gene (pfl) encoding pyruvate formate lyase and a gene (ldh) encoding lactate dehydrogenase. These deletion strains are preferably deletion strains of Escherichia coli.

When the “bacteria which are deficient in a function of one or more genes selected from the group consisting of ldh, ppc, pox, and pfl” are used as bacteria, it is possible to efficiently direct a saccharide added as a starting substance to production of pyruvic acid or pyruvic acid derivatives, as explained in the section of “2-1. First embodiment.”

In addition, the bacteria substantially do not proliferate when they are put under anaerobic conditions, and therefore, a saccharide can be efficiently used as a raw material for production of substances, instead of being used as an energy source for the proliferation.

In addition, when the bacteria are put in the presence of a nitrate ion, the nitrate ion is reduced to a nitrite ion by Reaction Formula (1), and NADH can be consumed during this reaction.

NO₃⁻+NADH+H⁺→NO₂⁻+NAD⁺H₂O (1)

Through this reaction, in the bacteria, NADH generated in the process of producing pyruvic acid from glucose is consumed, and the accumulation of NADH in bacterial cells is removed. Accordingly, it is possible to promote incorporation of a saccharide into bacterial cells and to promote the reaction of producing pyruvic acid from the saccharide.

Accordingly, according to the second embodiment, when the bacteria deficient in a function of a specific gene are incubated under anaerobic conditions in the presence of a saccharide and a nitrate ion, the bacteria can efficiently produce pyruvic acid or pyruvic acid derivatives from the saccharide.

### 3. Preferred Embodiments

Preferred embodiments of the present invention are collectively described below.

[A1] A method for producing a target substance from a starting substance via an NADH-accumulating reaction pathway, the method including:

incubating bacteria under aerobic conditions; and

subsequently incubating the bacteria under anaerobic conditions in the presence of the starting substance and a nitrate ion to produce the target substance.

[A2] The method according to [A1], in which the incubation of the bacteria in the presence of the starting substance and the nitrate ion is performed by incubating the bacteria in a culture solution containing the starting substance and the nitrate ion.
[A3] The method according to [A1] or [A2], in which the incubation of the bacteria in the presence of the starting substance and the nitrate ion is performed by incubating the bacteria in a culture solution into which the starting substance and a nitrate salt are added.
[A4] The method according to [A3], in which the nitrate salt is ammonium nitrate, sodium nitrate, potassium nitrate, or a combination thereof.
[A5] The method according to [A3] or [A4], in which the nitrate salt is added such that a concentration thereof in the culture solution is 10 to 500 mM, is preferably 100 to 500 mM, and is more preferably 200 to 300 mM.
[A6] The method according to any one of [A1] to [A5], in which the bacteria are aerobic bacteria.
[A7] The method according to any one of [A1] to [A6], in which the bacteria are coryneform bacteria, are preferably bacteria of the genus Corynebacterium, and are more preferably Corynebacterium glutamicum.
[A8] The method according to any one of [A1] to [A6], in which the bacteria are Escherichia coli.
[A9] The method according to any one of [A1] to [A8], in which, the bacteria are deficient in a function of one or more genes selected from the group consisting of a gene encoding lactate dehydrogenase, a gene encoding phosphoenolpyruvate carboxylase, a gene encoding pyruvate oxidase, and a gene encoding pyruvate formate lyase.
[A10] The method according to any one of [A1] to [A9], in which the starting substance is a saccharide.
[A11] The method according to any one of [A1] to [A10], in which the starting substance is a saccharide, and the target substance is “pyruvic acid” or a “compound biosynthesized through a glycolytic metabolite serving as a metabolic intermediate.”
[A12] The method according to [A10] or [A11], in which the saccharide is a monosaccharide such as fructose or glucose, a disaccharide such as sucrose, a polysaccharide such as a polysaccharide containing glucose as a constituent monosaccharide, or a saccharified solution obtained by treating a plant (for example, non-edible agricultural waste or energy crops) with a saccharification enzyme.
[A13] The method according to any one of [A10] to [A12], in which the saccharide is glucose.
[A14] The method according to any one of [A1] to [A13], further including reacting a nitrite ion, which is generated by reduction of the nitrate ion by the bacteria, with hydrogen peroxide such that the nitrite ion is oxidized to a nitrate ion.
[A15] The method according to any one of [A1] to [A14], in which the incubation of the bacteria under anaerobic conditions is performed in a state in which the bacteria are suspended in the culture solution at a high density.
[A16] The method according to any one of [A1] to [A15], in which the incubation of the bacteria under anaerobic conditions is performed in a state in which the bacteria are suspended in the culture solution such that a weight % of wet bacterial cells of the bacteria is within a range of 1 to 50 (w/v) %, and is preferably within in a range of 3 to 30 (w/v) %.
[A17] The method according to any one of [A1] to [A16], further including recovering the produced target substance.
[A18] The method according to any one of [A1] to [A17],

in which the NADH-accumulating reaction pathway is the following reaction pathway:

a reaction pathway which includes a reaction involving NADH production but does not include a reaction involving NADH consumption; or

a reaction pathway which includes a reaction involving NADH production and a reaction involving NADH consumption, and in which a total number of NADH molecules produced in the reaction involving NADH production is greater than a total number of NADH molecules consumed in the reactions involving NADH consumption.

[B1] A method for producing, from a saccharide via an NADH-accumulating reaction pathway, pyruvic acid or a compound biosynthesized through pyruvic acid serving as a metabolic intermediate, the method including:

incubating bacteria under aerobic conditions; and

subsequently incubating the bacteria under anaerobic conditions in the presence of a saccharide and a nitrate ion to produce pyruvic acid or a compound biosynthesized through pyruvic acid serving as a metabolic intermediate.

[B2] The method according to [B1], in which, the bacteria are deficient in a function of one or more genes selected from the group consisting of a gene encoding lactate dehydrogenase, a gene encoding phosphoenolpyruvate carboxylase, a gene encoding pyruvate oxidase, and a gene encoding pyruvate formate lyase.
[B3] A method for producing, from a saccharide via an NADH-accumulating reaction pathway, pyruvic acid or a compound biosynthesized through pyruvic acid serving as a metabolic intermediate, the method including:

incubating bacteria under anaerobic conditions in the presence of a saccharide and a nitrate ion to produce pyruvic acid or a compound biosynthesized through pyruvic acid serving as a metabolic intermediate, where the bacteria are deficient in a function of one or more genes selected from the group consisting of a gene encoding lactate dehydrogenase, a gene encoding phosphoenolpyruvate carboxylase, a gene encoding pyruvate oxidase, and a gene encoding pyruvate formate lyase.

[B4] The method according to any one of [B1] to [B3], wherein pyruvic acid is produced.
[B5] The method according to any one of [B1] to [B3], wherein the compound biosynthesized through pyruvic acid serving as a metabolic intermediate is produced.
[B6] The method according to [B1], [B2], [B3], or [B5], in which the compound biosynthesized through pyruvic acid serving as a metabolic intermediate is acetic acid.
[B7] The method according to any one of [B1] to [B6], in which the bacteria are a deletion strain deficient in a gene encoding lactate dehydrogenase.
[B8] The method according to any one of [B1] to [B6], in which the bacteria are a deletion strain deficient in a gene encoding lactate dehydrogenase and a gene encoding phosphoenolpyruvate carboxylase.
[B9] The method according to any one of [B1] to [B6], in which the bacteria are a deletion strain deficient in a gene encoding lactate dehydrogenase, a gene encoding phosphoenolpyruvate carboxylase, and a gene encoding pyruvate oxidase.
[B10] The method according to any one of [B1] to [B9], in which the bacteria are aerobic bacteria, are preferably coryneform bacteria, are more preferably bacteria of the genus Corynebacterium, and are even more preferably Corynebacterium glutamicum.
[B11] The method according to any one of [B1] to [B6], in which the bacteria are a deletion strain deficient in a gene encoding pyruvate formate lyase.
[B12] The method according to any one of [B1] to [B6], in which the bacteria are a deletion strain deficient in a gene encoding pyruvate formate lyase and a gene encoding lactate dehydrogenase.
[B13] The method according to any one of [B1] to [B6], [B11], and [B12], in which the bacteria are aerobic bacteria, and are preferably Escherichia coli.
[B14] The method according to any one of [B1] to [B13], in which the incubation of the bacteria in the presence of the saccharide and the nitrate ion is performed by incubating the bacteria in a culture solution containing the saccharide and the nitrate ion.
[B15] The method according to any one of [B1] to [B14], in which the incubation of the bacteria in the presence of the saccharide and the nitrate ion is performed by incubating the bacteria in a culture solution into which the saccharide and a nitrate salt are added.
[B16] The method according to [B15], in which the saccharide is added such that a concentration thereof in the culture solution is 0.1 to 40 (w/v) %, and is preferably 1 to 20 (w/v) %.
[B17] The method according to [B15] or [B16], in which the nitrate salt is added such that a concentration thereof in the culture solution is 10 to 500 mM, is preferably 100 to 500 mM, and is more preferably 200 to 300 mM.
[B18] method according to any one of [B1] to [B17], in which the saccharide is a monosaccharide such as fructose or glucose, a disaccharide such as sucrose, a polysaccharide such as a polysaccharide containing glucose as a constituent monosaccharide, or a saccharified solution obtained by treating a plant (for example, non-edible agricultural waste or energy crops) with a saccharification enzyme.
[B19] The method according to any one of [B1] to [B18], in which the saccharide is glucose.
[B20] The method according to any one of [B1] to [B19], in which the nitrate ion is added into the culture solution in the form of a nitrate salt, and the nitrate salt is ammonium nitrate, sodium nitrate, potassium nitrate, or a combination thereof.
[B21] The method according to any one of [B1] to [B20], further including reacting a nitrite ion, which is generated by reduction of the nitrate ion by the bacteria, with hydrogen peroxide such that the nitrite ion is oxidized to a nitrate ion.
[B22] The method according to any one of [B1] to [B21], in which the incubation of the bacteria under anaerobic conditions is performed in a state in which the bacteria are suspended in the culture solution at a high density.
[B23] The method according to any one of [B1] to [B22], in which the incubation of the bacteria under anaerobic conditions is performed in a state in which the bacteria are suspended in the culture solution such that a weight % of wet bacterial cells of the bacteria is within a range of 1 to 50 (w/v) %, and is preferably within in a range of 3 to 30 (w/v) %.
[B24] The method according to any one of [B1] to [B23], further including recovering the produced pyruvic acid or compound biosynthesized through pyruvic acid serving as a metabolic intermediate.
[B25] The method according to any one of [B1] to [B24],

in which the NADH-accumulating reaction pathway is the following reaction pathway:

a reaction pathway which includes a reaction involving NADH production but does not include a reaction involving NADH consumption; or

a reaction pathway which includes a reaction involving NADH production and a reaction involving NADH consumption, and in which a total number of NADH molecules produced in the reaction involving NADH production is greater than a total number of NADH molecules consumed in the reactions involving NADH consumption.

### Examples

1. Production of Deletion Strain (Δldh) of Ldh Gene

A deletion strain in Corynebacterium glutamicum ATCC 13032 was produced by the following method.

An SacB gene was amplified from pNIC-Bsa4 (Source Bioscience) using PrimeStar MAX (TAKARA) for an enzyme, using a T100™ Thermal Cycler (BIO-RAD), and using the following primers.

(SEQ ID NO: 1)
5′-GGGGAAGCTTGACGTCCACATATACCTGCC-3′
(SEQ ID NO: 2)
5′-ATTCGGATCCGTATCCACCTTTAC-3′

The obtained DNA fragment and a plasmid pHSG299 (TAKARA) were treated with restriction enzymes BamHI and HindIII, and then ligated using a DNA ligation Kit Ver. 2 (TAKARA). Thereby, pGE015 was obtained.

Furthermore, 1,000 bp upstream of an ldh gene was amplified using the following primers.

(SEQ ID NO: 3)
5′-GACGGCCAGTGAATTTTTCATACGACCACGGGCTA-3′
(SEQ ID NO: 4)
5′-GACAATCTTGTTACCGACGG-3′

1,000 bp downstream of an ldh gene was amplified using the following primers.

(SEQ ID NO: 5)
5′-GGTAACAAGATTGTCACCCTGCGCGAAATTCAGAA-3′
(SEQ ID NO: 6)
5′-TACCGAGCTCGAATTGAACTCACTGAAAAATGCTG-3′

The above amplifications were performed using a genomic DNA of ATCC 13032 as a template by the same method described above. The obtained DNA fragment was cloned into a vector, which was obtained by treating pGE015 with an EcoRI restriction enzyme, using an In-Fusion cloning kit (TAKARA), and this was designated as pGE033.

The plasmid pGE033 is a plasmid that contains a fragment in which an ldh gene has been deleted, and is an unreplicable plasmid within the genus Corynebacterium. According to a method of the electric pulse method (2500 V, 25 μF, 200Ω; Van der Rest et al. Appl. Microbiol Biotechnol 52, 541-545, 1999), the plasmid pGE033 was introduced into Corynebacterium glutamicum strain ATCC 13032, and applied to an A-agar medium (1 L [Composition: urea: 2 g, (NH₄)₂SO₄: 7 g, KH₂PO₄: 0.5 g, K₂HPO₄: 0.5 g, MgSO₄.7H₂O: 0.5 g, FeSO₄.7H₂O: 6 mg, MnSO₄.nH₂O: 4.2 mg, D-biotin: 200 μg, thiamine hydrochloride: 200 μg, yeast extract: 2 g, casamino acid: 7 g, glucose: 20 g, and agar: 16 g was dissolved in 1,000 ml distilled water (pH 6.6)]) containing 25 μg/ml of kanamycin.

A growth strain proliferated on the A-agar medium containing 50 μg/ml of kanamycin is a strain in which pGE033 has undergone one-point homologous recombination with a wild-type ldh gene on the chromosome and thereby the entire plasmid has been integrated into the genomic DNA. When this strain was applied to an LB agar medium (Bacto Peptone: 10 g, yeast extract: 5 g, sodium chloride: 10 g, agar: 16 g/l L) containing 10% sucrose, bacterial cells having the SacB gene could not survive because they stored toxic substances in cells, and only the strain from which the SacB gene had been deleted survived. In this case, when the plasmid has been deleted in the original form of pGE033, it returns to the wild-type strain ATCC 13032, but when the plasmid has been deleted while carrying an ldh gene, a deletion strain deficient in the ldh gene remains.

Selection of a deletion strain deficient in an ldh gene was carried out by a PCR method (colony PCR) using a colony as a template, and using the following primers.

(SEQ ID NO: 3)
5′-GACGGCCAGTGAATTTTTCATACGACCACGGGCTA-3′
(SEQ ID NO: 6)
5′-TACCGAGCTCGAATTGAACTCACTGAAAAATGCTG-3′

Since a DNA fragment from which a 2-kb ldh gene is deleted would have been obtained for a deletion strain, this condition was confirmed by agarose gel electrophoresis (Molecular Cloning, Sambrook et al., 1989 Cold Spring Harbor Laboratory Press), and a colony in which a deleted fragment was confirmed was selected as a deletion strain (ATCC 13032 Δldh).

2. Production of Deletion Strain (ΔLdh Δppc) Deficient in Ldh Gene and Ppc Gene Deletion of a ppc gene was carried out as follows according to the same method as that for the deletion of the ldh gene. 1,000 bp upstream of a ppc gene was amplified using the following primers.

(SEQ ID NO: 7)
5′-CCATGATTACGAATTCGGGAAACTTTTTTAAGAAA-3′
(SEQ ID NO: 8)
5′-TAACTACTTTAAACACTCTT-3′

1,000 bp downstream of a ppc gene was amplified using the following primers.

(SEQ ID NO: 9)
5′-TGTTTAAAGTAGTTATCCAGCCGGCTGGGTAGTAC-3′
(SEQ ID NO: 10)
5′-TACCGAGCTCGAATTGAAGTATTCAAGGGGATTTC-3′

The above amplifications were performed using a genomic DNA of ATCC 13032 as a template. The obtained DNA fragment was cloned into a vector, which was obtained by treating pGE015 with an EcoRI restriction enzyme, using an In-Fusion cloning kit (TAKARA), and this was designated as pGE020. The plasmid pGE020 was introduced into strain ATCC 13032 Δldh according to the method of the electric pulse method, and a deletion strain deficient in the ldh gene and the ppc gene (ATCC 13032 Δldh Δppc) was obtained by the same method as described above.

3. Production of Deletion Strain Deficient in Ldh Gene, Ppc Gene, and Pox Gene (ΔLdh Δppc Δpox)

Deletion of a pox gene was carried out as follows according to the same method as that for the deletion of the ldh gene. 1,000 bp upstream of a pox gene was amplified using the following primers.

(SEQ ID NO: 11)
5′-GACGGCCAGTGAAAACGTTAATGAGGAAAACCG-3′
(SEQ ID NO: 12)
5′-AATTAATTGTTCTGCGTAGC-3′

1,000 bp downstream of a pox gene was amplified using the following primers.

(SEQ ID NO: 13)
5′-GCAGAACAATTAATTCTCGAGTCGAACATAAGGAATATTCC-3′
(SEQ ID NO: 14)
5′-TACCGAGCTCGAATTTTCCAGGTACGGAAAGTGCC-3′

The above amplifications were performed using a genomic DNA of ATCC 13032 as a template. The obtained DNA fragment was cloned into a vector, which was obtained by treating pGE15 with an EcoRI restriction enzyme, using an In-Fusion cloning kit (TAKARA), and this was designated as pGE191. The plasmid pGE191 was introduced into strain ATCC 13032 Δldh Δppc according to the method of the electric pulse method, and a deletion strain deficient in the ldh gene, the ppc gene, and the pox gene (ATCC 13032 Δldh Δppc ΔpoxB) was obtained by the same method as described above.

1. Production of Deletion Strain (ΔpflB) Deficient in pflB Gene

Production of a deletion strain of Escherichia coli was performed according to a method of Datsenko and Wanner (Proc Natl Acad Sci USA 2000, 97: 6640-6645). A strain obtained by transforming Escherichia coli strain BW 25113 with pKD46 (Life Science Market) was cultured at 30° C. in a medium obtained by adding 10 mM of arabinose into 100 ml of an LB medium until OD600 reached around 0.6. The bacterial cells were washed 3 times with 10% glycerol, and finally suspended in 1 ml of 10% glycerol. For production of a deletion strain deficient in a pflB gene, a gene fragment, which had been amplified using pKD13 (Life Science Market) as a template and using the following primers, was used.

(SEQ ID NO: 15)
5′-CGAAGTACGCAGTAAATAAAAAATCCACTTAAGAAGGTAGGTGTTAC
ATGATTCCGGGGATCCGTCGACC-3′
(SEQ ID NO: 16)
5′-TTTTACTGTACGATTTCAGTCAAATCTAATTACATAGATTGAGTGAA
GGTTGTAGGCTGGAGCTGCTTCG-3′

The PCR product was purified using NucleoSpin Gel and PCR Clean-Up (TAKARA).

10 μl of the PCR product prepared above was added into 150 μl of the competent cells prepared above to perform gene transfer by an electric pulse method (2500 V, 25 μF, 200Ω). A growth strain proliferated on an LB agar medium containing 50 μg/ml of kanamycin was selected. Deletion was confirmed by culturing bacterial cells, analyzing the supernatant with an organic acid, and checking that formic acid was not being produced anymore. Regarding organic acid measurement, it was identified by HPLC analysis using a column of TSKgel OApak (TOSOH).

A strain obtained by transforming strain BW 25113 ΔpflB::Km with pCP20 (Life Science Market) at 30° C. was cultured at 42° C. by streaking it on an LB plate not containing a drug. pCP20 is designed to express Flp recombinase in which cassettes of a kanamycin-resistant gene have been deleted at a high temperature. A colony sensitive to kanamycin was selected from the plate cultured at 42° C., and this was designated as a deletion strain deficient in a pflB gene (BW 25113 ΔpflB).

5. Production of Deletion Strain (ΔpflB ΔLdh) Deficient in pflB Gene and ldhA Gene

Competent cells were prepared from a strain obtained by transforming strain BW 25113 ΔpflB with pKD46 (Life Science Market) by the above-described method, and a DNA fragment in which ldhA had been replaced by a kanamycin-resistant gene was transformed by the above-described method. The DNA fragment in which ldhA had been replaced by the kanamycin-resistant gene was prepared by performing a PCR reaction using pKD13 as a template and using the following primers.

(SEQ ID NO: 17)
5′-CTGCCGGGAAATGACTGTCCGCAAGGAAAAGAGTGTGAGGAAAAACA
ATGATTCCGGGGATCCGTCGACC-3′
(SEQ ID NO: 18)
5′-AAAGAAGTAATGTTTTCTTCATCATCACCTCAGAAAAGATCGCTACG
AGTTGTAGGCTGGAGCTGCTTCG-3′

The PCR product was purified using NucleoSpin Gel and PCR Clean-Up (TAKARA). Deletion of the transformant was confirmed by culturing bacterial cells, analyzing the supernatant with an organic acid, and checking that lactic acid was not being produced anymore. Accordingly, a deletion strain (BW 25113 ΔpflB Δldh) deficient in the pflB gene and the ldhA gene was obtained.

6. Production Example 1 for Pyruvic Acid and Pyruvic Acid Derivatives

Corynebacterium glutamicum ATCC 13032, ATCC 13032 Δldh, ATCC 13032 Δldh Δppc, and ATCC 13032 Δldh Δppc ΔpoxB were respectively pre-cultured (test tubes) in 5 ml of an A medium, and they were shake-cultured at 33° C. and 180 rpm for 20 hours in 500 ml of an A medium put in a 2-L flask (proliferation culture).

After the culturing, the cells were centrifuged to remove the culture solution and were suspended in 50 ml of a BT solution ((NH₄)₂SO₄: 7 g, KH₂PO₄: 0.5 g, K₂HPO₄: 0.5 g, MgSO₄.7H₂O: 0.5 g, FeSO₄.7H₂O: 6 mg, MnSO₄.nH₂O: 4.2 mg/L), and a concentration was adjusted so that an OD reached around 100 (weight of wet bacterial cells 12 (w/v) %). The cells were transferred to a 100-ml medium bottle containing a stir bar, and 2.5 ml of 50% glucose, 2.5 ml of 6M NaNO₃ (denoted as “+NaNO₃” in Table 1), or 2.5 ml of water (denoted as “—NaNO₃” in Table 1) was further added thereinto. NaNO₃ was 250 mM in the culture solution.

This medium bottle was put in a constant-temperature tank at 33° C., allowed to stand, and reacted with stirring (production culture). A pH was adjusted to pH 6.5 with 2.5 N KOH using a pH controller.

After one hour, 0.5 ml of the reaction solution was taken and centrifuged to obtain a supernatant, and an amount of glucose consumed and an amount of organic acid produced were measured.

The measurement of glucose was performed using a biosensor BF-7 (Oji Scientific Instruments Co., Ltd.). The measurement of organic acid was performed by attaching a column of TSKgel OA-pack (TOSOH CORPORATION) to an organic acid analysis HPLC apparatus (Shimadzu Corporation).

The results are shown in the table below.

TABLE 1

| | Glucose | | | | |
|---|---|---|---|---|---|
| | consumed | Organic acid produced (mM) | | | |
| | in 1 hour | Lactic | Succinic | Acetic | Pyruvic |
| | (mM) | acid | acid | acid | acid |
| ATCC 13032 | 76.1 | 129.1 | 15.8 | 0.0 | 0.0 |
| (−NaNO₃) | | | | | |
| ATCC 13032 | 96.6 | 106.8 | 13.1 | 29.9 | 20.1 |
| (+NaNO₃) | | | | | |
| Δldh (−NaNO₃) | 3.4 | 0.0 | 4.4 | 7.3 | 0.0 |
| Δldh (+NaNO₃) | 79.4 | 0.0 | 38.9 | 51.1 | 41.3 |
| Δldh Δppc | 0.0 | 0.0 | 0.0 | 0.0 | 0.0 |
| (−NaNO₃) | | | | | |
| Δldh Δppc | 46.2 | 0.0 | 9.2 | 22.7 | 48.2 |
| (+NaNO₃) | | | | | |
| Δldh Δppc ΔpoxB | 0.0 | 0.0 | 0.0 | 0.0 | 0.0 |
| (−NaNO₃) | | | | | |
| Δldh Δppc ΔpoxB | 53.6 | 0.0 | 5.1 | 0.0 | 97.6 |
| (+NaNO₃) | | | | | |

As shown in Table 1, in the cases of using any coryneform bacteria of ATCC 13032, the deletion strain (Δldh) deficient in an ldh gene, the deletion strain (Δldh Δppc) deficient in an ldh gene and a ppc gene, and the deletion strain (Δldh Δppc Δpox) deficient in an ldh gene, a ppc gene, and a pox gene, an amount of pyruvic acid produced and an amount of acetic acid produced could be increased by adding sodium nitrate, as compared to a case in which sodium nitrate was not added. However, only Δldh Δppc Δpox could not produce acetic acid by adding sodium nitrate, because a pox gene was deleted therefrom. This result shows that a pathway (pta-ackA, ctfA) that produces acetic acid from glucose via acetyl-CoA does not act, and that pyruvate reductase (AceEF, Ldh) does not act even in consideration of the condition in which more than 90% of carbon was emitted as pyruvic acid. Removing the accumulation of NADH can also be performed by blowing air (oxygen), but in this case, pyruvate reductase acts and the TCA cycle also acts. On the other hand, removing the accumulation of NADH by adding nitrate salts is an extremely excellent method from the viewpoint that the accumulation of NADH can be removed without causing pyruvate reductase to act.

The following can be understood from pieces of data when sodium nitrate was added (refer to pieces of data for ATCC 13032 (+NaNO₃), Δldh (+NaNO₃), Δldh Δppc (+NaNO₃), and Δldh Δppc ΔpoxB (+NaNO₃) in Table 1). When an ldh gene was deleted from ATCC 13032, this could further increase an amount of pyruvic acid produced. When a ppc gene was deleted from the deletion strain (Δldh) deficient in the ldh gene, this could further increase an amount of pyruvic acid produced. When a pox gene was deleted from the deletion strain (Δldh Δppc) deficient in the ldh gene and the ppc gene, this could further increase an amount of pyruvic acid produced.

7. Production Example 2 for Pyruvic Acid and Pyruvic Acid Derivatives

In Production Example 1, the production culture was carried out after the culture solution was exchanged after the proliferation culture, but in Production Example 2, production culture was carried out after the proliferation culture, without exchanging the culture solution, but by adding glucose and sodium nitrate.

ATCC 13032 Δldh Δppc ΔpoxB was pre-cultured (test tubes) in 5 ml of an A medium, and it was shake-cultured at 33° C. and 180 rpm for 20 hours in 50 ml of an A medium put in a 500-mL flask (proliferation culture).

After the culturing, without replacing the medium by a BT solution, the cells were transferred to a 100-ml medium bottle containing a stir bar, and 1 ml of 50% glucose, 1 ml of 6M NaNO₃ (denoted as “+NaNO₃” in Table 2), or 1 ml of water (denoted as “—NaNO₃” in Table 2) was further added thereinto. NaNO₃ was 100 mM in the culture solution. This medium bottle was put in a constant-temperature tank at 33° C., allowed to stand, and reacted with stirring (production culture). A pH was adjusted to pH 6.5 with 2.5 N KOH using a pH controller.

After eight hours, 0.5 ml of the reaction solution was taken and centrifuged to obtain a supernatant, and an amount of glucose consumed and an amount of organic acid produced were measured.

The results are shown in the table below.

TABLE 2

| | Glucose | | | | |
|---|---|---|---|---|---|
| | consumed | Organic acid produced (mM) | | | |
| | in 8 hours | Lactic | Succinic | Acetic | Pyruvic |
| | (mM) | acid | acid | acid | acid |
| Δldh Δppc ΔpoxB | 14.6 | 0.0 | 7.0 | 10.4 | 1.3 |
| (−NaNO₃) | | | | | |
| Δldh Δppc ΔpoxB | 56.6 | 0.0 | 9.0 | 0.0 | 62.0 |
| (+NaNO₃) | | | | | |

As shown in Table 2, in the deletion strain (Δldh Δppc Δpox) deficient in an ldh gene, a ppc gene, and a pox gene, an amount of pyruvic acid produced could be increased by adding sodium nitrate, as compared to a case in which sodium nitrate was not added. Δldh Δppc Δpox could not produce acetic acid by adding sodium nitrate, because a pox gene was deleted therefrom.

Based on this result, it was found that it is not always required to exchange a medium between proliferation culture and production culture.

8. Production Example 3 for Pyruvic Acid and Pyruvic Acid Derivatives

Escherichia coli BW 25113, BW 25113 ΔpflB, and BW 25113 ΔpflB ΔldhA were respectively pre-cultured (test tubes) in 5 ml of an LB medium, and they were shake-cultured at 37° C. and 180 rpm for 20 hours in 100 ml of a terrific medium (Bacto Peptone: 12 g, yeast extract: 24 g, glycerol: 4 ml, KH₂PO₄: 2.31 g, K₂HPO₄: 12.54 g) put in a 500-mL flask (proliferation culture).

After the culturing, the cells were centrifuged to remove the culture solution and were suspended in 50 ml of a BT solution, and a concentration was adjusted so that an OD reached around 24 (weight of wet bacterial cells 3 (w/v) %). The cells were transferred to transferred to a 100-ml medium bottle containing a stir bar, and 1 ml of 50% glucose, 1 ml of 6M NaNO₃ (denoted as “+NaNO₃” in Table 3), or 1 ml of water (denoted as “—NaNO₃” in Table 3) was further added thereinto. NaNO₃ was 120 mM in the culture solution.

This medium bottle was put in a constant-temperature tank at 37° C., allowed to stand, and reacted with stirring (production culture). A pH was adjusted to pH 6.5 with 2.5 N KOH using a pH controller.

After four hours, 0.5 ml of the reaction solution was taken and centrifuged to obtain a supernatant, and an amount of glucose consumed and an amount of organic acid produced were measured.

The results are shown in the table below.

TABLE 3

| | Glucose | | | | | |
|---|---|---|---|---|---|---|
| | consumed | Organic acid produced (mM) | | | | |
| | in 4 hours | Formic | Lactic | Succinic | Acetic | Pyruvic |
| | (mM) | acid | acid | acid | acid | acid |
| BW 25113 | 82.4 | 91.8 | 25.1 | 8.5 | 46.7 | 0.0 |
| (−NaNO₃) | | | | | | |
| BW 25113 | 64.6 | 18.9 | 10.7 | 0.0 | 68.6 | 19.0 |
| (+NaNO₃) | | | | | | |
| ΔpflB | 109.6 | 0.0 | 156.5 | 18.1 | 0.0 | 0.0 |
| (−NaNO₃) | | | | | | |
| ΔpflB | 62.0 | 0.0 | 28.6 | 0.0 | 33.6 | 41.9 |
| (+NaNO₃) | | | | | | |
| ΔpflB | 16.8 | 0.0 | 0.0 | 0.0 | 0.0 | 16.4 |
| Δldh | | | | | | |
| (−NaNO₃) | | | | | | |
| ΔpflB | 62.7 | 0.0 | 0.0 | 0.0 | 18.0 | 95.5 |
| Δldh | | | | | | |
| (+NaNO₃) | | | | | | |

As shown in Table 3, in the cases of using any Escherichia coli of BW 25113, the deletion strain (ΔpflB) deficient in a pflB gene, and the deletion strain (ΔpflB Δldh) deficient in a pflB gene and a ldh gene, an amount of pyruvic acid produced and an amount of acetic acid produced could be increased by adding sodium nitrate, as compared to a case in which sodium nitrate was not added.

The following can be understood from pieces of data when sodium nitrate was added (refer to pieces of data for BW 25113 (+NaNO₃), ΔpflB (+NaNO₃), and ΔpflB Δldh (+NaNO₃) in Table 3). When a pflB gene was deleted from BW 25113, this could further increase an amount of pyruvic acid produced. When an ldh gene was deleted from the deletion strain (ΔpflB) deficient in the pflB gene, this could further increase an amount of pyruvic acid produced.

1. A method for producing a target substance from a starting substance via an NADH-accumulating reaction pathway, the method comprising:
(i) incubating bacteria under aerobic conditions; and
(ii) subsequently incubating the bacteria under anaerobic conditions in the presence of the starting substance and a nitrate ion to produce the target substance.

2. The method according to claim 1, wherein the bacteria are deficient in a function of one or more genes selected from the group consisting of a gene encoding lactate dehydrogenase, a gene encoding phosphoenolpyruvate carboxylase, a gene encoding pyruvate oxidase, and a gene encoding pyruvate formate lyase.

3. The method according to claim 1, further comprising reacting a nitrite ion, which is generated by reduction of the nitrate ion by the bacteria, with hydrogen peroxide such that the nitrite ion is oxidized to a nitrate ion.

4. A method for producing, from a saccharide via an NADH-accumulating reaction pathway, pyruvic acid or a compound biosynthesized through pyruvic acid serving as a metabolic intermediate, the method comprising:
(i) incubating bacteria under aerobic conditions; and
(ii) subsequently incubating the bacteria under anaerobic conditions in the presence of a saccharide and a nitrate ion to produce pyruvic acid or a compound biosynthesized through pyruvic acid serving as a metabolic intermediate.

5. The method according to claim 4, wherein the bacteria are deficient in a function of one or more genes selected from the group consisting of a gene encoding lactate dehydrogenase, a gene encoding phosphoenolpyruvate carboxylase, a gene encoding pyruvate oxidase, and a gene encoding pyruvate formate lyase.

6. A method for producing, from a saccharide via an NADH-accumulating reaction pathway, pyruvic acid or a compound biosynthesized through pyruvic acid serving as a metabolic intermediate, the method comprising:
incubating bacteria under anaerobic conditions in the presence of a saccharide and a nitrate ion to produce pyruvic acid or a compound biosynthesized through pyruvic acid serving as a metabolic intermediate, where the bacteria are deficient in a function of one or more genes selected from the group consisting of a gene encoding lactate dehydrogenase, a gene encoding phosphoenolpyruvate carboxylase, a gene encoding pyruvate oxidase, and a gene encoding pyruvate formate lyase.

7. The method according to claim 4, wherein pyruvic acid is produced.

8. The method according to claim 4, wherein the compound biosynthesized through pyruvic acid serving as a metabolic intermediate is produced.

9. The method according to claim 4, wherein the compound biosynthesized through pyruvic acid serving as a metabolic intermediate is acetic acid.

10. The method according to claim 4, wherein the bacteria are a deletion strain deficient in a gene encoding lactate dehydrogenase.

11. The method according to claim 4, wherein the bacteria are a deletion strain deficient in a gene encoding lactate dehydrogenase and a gene encoding phosphoenolpyruvate carboxylase.

12. The method according to claim 4, wherein the bacteria are a deletion strain deficient in a gene encoding lactate dehydrogenase, a gene encoding phosphoenolpyruvate carboxylase, and a gene encoding pyruvate oxidase.

13. The method according to claim 4, wherein the bacteria are coryneform bacteria.

14. The method according claim 4, wherein the bacteria are a deletion strain deficient in a gene encoding pyruvate formate lyase.

15. The method according to claim 4, wherein the bacteria are a deletion strain deficient in a gene encoding pyruvate formate lyase and a gene encoding lactate dehydrogenase.

16. The method according to claim 4, wherein the bacteria are Escherichia coli.

17. The method according to claim 4, further comprising reacting a nitrite ion, which is generated by reduction of the nitrate ion by the bacteria, with hydrogen peroxide such that the nitrite ion is oxidized to a nitrate ion.

18. The method according to claim 1, wherein, in step (ii), the bacteria are incubated in a culture solution comprising from 10 mM to 500 mM of a nitrate salt, to produce the target substance.

19. The method according to claim 4, wherein, in step (ii), the bacteria are incubated in a culture solution comprising from 10 mM to 500 mM of a nitrate salt, to produce the pyruvic acid or the compound biosynthesized through pyruvic acid serving as a metabolic intermediate.

20. The method according to claim 6, wherein the bacteria are incubated in a culture solution comprising from 10 mM to 500 mM of a nitrate salt, to produce the pyruvic acid or the compound biosynthesized through pyruvic acid serving as a metabolic intermediate.


SEQUENCE LISTING (18 sequences)

SEQ ID NO: 1 - DNA, 30 bp, Artificial Sequence
  Forward primer for sacB gene
ggggaagcttgacgtccacatatacctgcc

SEQ ID NO: 2 - DNA, 24 bp, Artificial Sequence
  Reverse primer for sacB gene
attcggatccgtatccacctttac

SEQ ID NO: 3 - DNA, 35 bp, Artificial Sequence
  Forward primer for upper region of ldh gene
gacggccagtgaatttttcatacgaccacgggcta

SEQ ID NO: 4 - DNA, 20 bp, Artificial Sequence
  Reverse primer for upper region of ldh gene
gacaatcttgttaccgacgg

SEQ ID NO: 5 - DNA, 35 bp, Artificial Sequence
  Forward primer for lower region of ldh gene
ggtaacaagattgtcaccctgcgcgaaattcagaa

SEQ ID NO: 6 - DNA, 35 bp, Artificial Sequence
  Reverse primer for lower region of ldh gene
taccgagctcgaattgaactcactgaaaaatgctg

SEQ ID NO: 7 - DNA, 35 bp, Artificial Sequence
  Forward primer for upper region of ppc gene
ccatgattacgaattcgggaaacttttttaagaaa

SEQ ID NO: 8 - DNA, 20 bp, Artificial Sequence
  Reverse primer for upper region of ppc gene
taactactttaaacactctt

SEQ ID NO: 9 - DNA, 35 bp, Artificial Sequence
  Forward primer for lower region of ppc gene
tgtttaaagtagttatccagccggctgggtagtac

SEQ ID NO: 10 - DNA, 35 bp, Artificial Sequence
  Reverse primer for lower region of ppc gene
taccgagctcgaattgaagtattcaaggggatttc

SEQ ID NO: 11 - DNA, 33 bp, Artificial Sequence
  Forward primer for upper region of pox gene
gacggccagtgaaaacgttaatgaggaaaaccg

SEQ ID NO: 12 - DNA, 20 bp, Artificial Sequence
  Reverse primer for upper region of pox gene
aattaattgttctgcgtagc

SEQ ID NO: 13 - DNA, 41 bp, Artificial Sequence
  Forward primer for lower region of pox gene
gcagaacaattaattctcgagtcgaacataaggaatattcc

SEQ ID NO: 14 - DNA, 35 bp, Artificial Sequence
  Reverse primer for lower region of pox gene
taccgagctcgaattttccaggtacggaaagtgcc

SEQ ID NO: 15 - DNA, 70 bp, Artificial Sequence
  Forward primer for pflB gene deletion
cgaagtacgcagtaaataaaaaatccacttaagaaggtaggtgttacatgattccggggatccgtcgacc

SEQ ID NO: 16 - DNA, 70 bp, Artificial Sequence
  Reverse primer for pflB gene deletion
ttttactgtacgatttcagtcaaatctaattacatagattgagtgaaggttgtaggctggagctgcttcg

SEQ ID NO: 17 - DNA, 70 bp, Artificial Sequence
  Forward primer for ldhA gene deletion
ctgccgggaaatgactgtccgcaaggaaaagagtgtgaggaaaaacaatgattccggggatccgtcgacc

SEQ ID NO: 18 - DNA, 70 bp, Artificial Sequence
  Reverse primer for ldhA gene deletion
aaagaagtaatgttttcttcatcatcacctcagaaaagatcgctacgagttgtaggctggagctgcttcg